(19) United States
(12) Patent Application Publication
(10) Pub. No.: US 2022/0300807 A1
(43) Pub. Date: Sep. 22, 2022

(54) SYSTEMS AND METHODS FOR APPLYING A TRANSFORMER NETWORK TO SPATIAL DATA

(71) Applicant: Phronesis Artificial Intelligence, Inc., Daphne, AL (US)

(72) Inventor: William Carl SPAGNOLI, Marina Del Rey, CA (US)

(21) Appl. No.: 17/584,087

(22) Filed: Jan. 25, 2022

Related U.S. Application Data
(60) Provisional application No. 63/200,612, filed on Mar. 18, 2021.

(51) Int. Cl. G06N 3/08; G06N 3/063; G16C 20/50; G16C 20/70; G16C 20/20

(52) U.S. Cl. CPC G06N 3/08; G06N 3/063; G16C 20/50; G16C 20/70; G16C 20/20

(57) ABSTRACT

Systems and methods for a process for applying Transformer Neural Networks to Spatial Data comprising: Representing User Inputs in the form of one or more numeric matrices of one or more dimensions; Using one or more Transformer Neural Networks to predict a molecule's binding affinity with a protein receptor and/or other molecular attributes for one or more molecules.

### CROSS-REFERENCE TO RELATED APPLICATIONS

This application claims benefit of U.S. provisional patent application Ser. No. 63/200,612, filed Mar. 18, 2021, which is herein incorporated by reference.

### FIELD

The embodiments hereof relate to the creation of novel molecules

### BACKGROUND

There exists a need to discover molecules capable of use for many applications, and in particular, as candidates for the prevention or treatment of disease, including infectious diseases. For example, viruses are known to attach to, and infect, cells by the connection of a cell ligand to virus receptor. The receptor mimics some other beneficial connection with the cell, and is thus able to attach to the cell and use the cell to replicate itself. To prevent the virus from accomplishing this, a means of blocking the virus receptor so that it cannot attach to the cell can be used.

### SUMMARY

In one aspect, a method of determining the applicability of a candidate molecule for the treatment of a disease, wherein the disease is caused by a multi-atom agent, for example an infectious agent, an autoimmune agent, cancerous cells, and the like, includes identifying the location of the different atomic species of the candidate molecule in three dimensions, identifying the location of the different atomic species in the multi atom agent, determining the likelihood that the candidate molecule would bind to the multi atom agent, and identifying the suitability of the candidate molecule for a pharmaceutical application based upon at least one candidate molecule property in addition to the likelihood that the candidate molecule would bind to the multi atom agent. In an aspect, the multi atom agent is a target receptor and the likelihood that the target molecule would bind to the multi atom agent is the likelihood that the target molecule would bind to the target receptor.

In another aspect a method for evaluation of candidate molecules for pharmaceutical application includes creating a vector representation of both a molecule and a target receptor's three-dimensional structure, creating a high-dimensional embedding of this vector representation, adding positional encoding to the embedded representations, inputting these representations into one or more Transformer Neural Networks consisting of either one or more encoder blocks, decoder blocks, or both, and predicting one or more molecular attributes based upon the output from the one or more Transformer Neural Networks including, but not limited to the binding affinity of said molecule with the target receptor.

### BRIEF DESCRIPTION OF THE DRAWINGS

The components in the figures are not necessarily to scale, emphasis instead being placed upon illustrating the principals of the invention. Like reference, numerals designate corresponding parts throughout the different views. Embodiments are illustrated by way of example and not limitation in the figures of the accompanying drawings, in which:

FIG. 1 depicts a top-level functional block diagram of a computing system environment;

FIG. 2 depicts components in communication with a processor of the computing system of FIG. 1;

FIG. 3 depicts a flow diagram of applying Transformer Neural Networks (800) to Spatial Data, also known as the “Fast, Accurate, and Versatile, In-Silico Measurement” (FAVIM) process;

FIG. 4 depicts a flow diagram of a molecular representation;

FIG. 5 depicts a flow diagram of a Single Molecular Attribute Representation (500);

FIG. 6 depicts a flow diagram of a three-dimensional voxel map representation;

FIG. 7 depicts a flow diagram of an input preparation process;

FIG. 8 depicts a flow diagram of the internal architecture of one of the Transformer Neural Networks (800) within the Transformer Neural Network Component (1200);

FIG. 9 depicts a flow diagram of additional internal componentry of one of the Transformer Neural Networks (800) within the Transformer Neural Network Component (1200);

FIG. 10 depicts a flow diagram of a Multi-Head Attention Layer (1000) of one of the Transformer Neural Networks (800) within the Transformer Neural Network Component (1200);

FIG. 11 depicts a flow diagram of an Input Embedding (901) for a Three-Dimensional Voxel Map (600).

FIG. 12 depicts a schematic of a training process for a Transformer Neural Network Component (1200) within an embodiment using a Contrastive Language-In-Silico Pre-training (CLISP) design.

FIG. 13 depicts a schematic of a Transformer Neural Network Component (1200) within an embodiment using a Contrastive Language-In-Silico Pre-training (CLISP) design.

FIG. 14 depicts a flow diagram of an Encoder Block (803) of one of the Transformer Neural Networks (800) within the Transformer Neural Network Component (1200);

FIG. 15 depicts a flow diagram of an Encoder Block (803) of one of the Transformer Neural Networks (800) within the Transformer Neural Network Component (1200);

FIG. 16 depicts a block diagram of the system.

FIG. 17 shows a high-level block diagram and process of a computing system for implementing an embodiment of the system and process;

FIG. 18 shows a block diagram and process of an exemplary system in which an embodiment may be implemented; and

FIG. 19 depicts a cloud computing environment for implementing an embodiment of the system and process disclosed herein.

### DETAILED DESCRIPTION

In One aspect, the described technology concerns one or more methods, systems, apparatuses, and mediums storing processor-executable process steps of high-accuracy prediction of molecular attributes, including, but not limited to the binding affinity of said molecule with a chosen target receptor. In one embodiment, this technology is capable of accurately predicting a novel molecule's binding affinity with a given target receptor simultaneously along with a wide array of other desired drug properties. In another aspect, the described technology concerns one or more methods, systems, apparatuses, and mediums storing processor-executable process steps of automated targeted molecular design allowing a user or users to design molecules of any desired traits, and providing detailed metrics for the new molecules to the user or users. In one embodiment, a targeted molecular design application may automatically provide organized, easy to understand, and sortable measurements of newly generated molecules, allowing the user to immediately view side-by-side comparisons of the relevant properties in new molecules for which the user has requested an evaluation or understanding of.

Embodiments hereof relate generally to determining correlative properties between at least two different multi-atom structures, such as a molecule and a target receptor of a virus. In one aspect, this includes application of a transformer neural network to spatial data related to the location of, for example, individual atoms in the multi-atom structures and determining the affinity of one of the multi-atom structures to bind to another of the multi-atom structures, for example, of a molecule or ligand thereof to bind to a receptor target. In another aspect, this additionally includes a determination of other molecular properties such as size, solubility or other properties related to the ability of the molecule to function as a pharmaceutical, for example as an oral or injectable pharmaceutical.

Detecting structure-dependent molecular properties from three-dimensional spatial data or from a three dimensional model of the molecule is an important predictive drug screening task with a wide range of applications, including, but not limited to, the prediction of a molecule's binding affinity with a given protein receptor target, molecular weight, or solubility. For example, throughout the current global pandemic caused by the novel virus, Covid-19, there is a global effort to find an effective drug to combat the deadly disease. The Covid-19 Spike protein has been identified as a key receptor target for small-molecule inhibitor drugs, as the ability to inhibit this receptor removes the virus' ability to enter human cells. To understand the efficacy of a potential drug candidate to inhibit Covid-19, medical or pharmacological professionals must simultaneously determine:
a) the candidate drug's binding affinity with a target protein, such as a target protein on a virus, to ensure it is able to inhibit the receptor, for example by binding thereto so as to prevent the target protein from binding to a living cell;
b) the candidate drug's molecular weight to ensure the candidate drug can pass through the cell membrane to be absorbed into the human body;
c) the candidate drug's solubility to ensure the candidate drug can be consumed orally;
d) and many other necessary molecular attributes.

When searching for a new drug molecule to treat a novel infectious disease amid a global pandemic, there is an urgent priority to identify promising new candidate drugs, resulting in the measurement of countless drug candidates which measurement requires high speed screening, accuracy, and versatility. However, there is currently limited availability of screening process and knowledge to perform this screening at high speed while evaluating a new drug candidate for efficacy where each of the desired properties of the new drug candidate can be readily determined.

Additionally, viruses mutate, and where left to replicate unabated in a host, the likelihood of mutation increases. Likewise, with respect to bacterial infections, as known bacterial infectious agents are exposed to the same pharmaceutical(s) over time, natural variants of the infectious agent can arise as a result of mutation, which are then selected for in vivo when they are exposed to a pharmaceutical known to effectively treat the non-mutated bacteria, when the pharmaceutical cannot effectively treat the genetic variant. As a result, infectious diseases caused by bacterial infections have developed antibiotic resistant strains, and there is a current need for a mechanism to rapidly screen multiple candidate antibiotics to identify candidate molecules that may be capable of effectively treating these resistant strains. In a similar fashion, cancer cells mutate, and there is a need to rapidly identify a suitable cell specific therapeutic agent to suppress or destroy these cells. Additionally, in autoimmune disease, there is a need to develop immune-suppressants or blockers to prevent the bodies immune system from attacking healthy cells and tissue.

In each case, the candidate molecule, and the infectious agent, are multi-atom structures having three dimensions, such that the ability of the candidate molecule to bind to the target, for example to a site on an infectious agent, to a cancer cell, or to an immune system agent is not simply a function of the atomic species present in the candidate molecule and the target infectious agent. For example, it may be known that a certain molecule can bind to a certain target receptor protein. But if the local topography of the target protein shrouds or partially projects from the target receptor location, the molecule may be of too large a size for the portion thereof binding to the target protein to physically reach the target receptor. Likewise, interatomic interactions between a potential binding molecule and a target receptor may also prevent a likely candidate molecule from binding to a target receptor.

The techniques introduced below may be implemented by programmable circuitry programmed or configured by software and/or firmware, or entirely by special-purpose circuitry, or in a combination of such forms. Such special-purpose circuitry (if any) can be in the form of, for example, one or more application-specific integrated circuits (ASICs), programmable logic devices (PLDs), field-programmable gate arrays (FPGAs), etc.

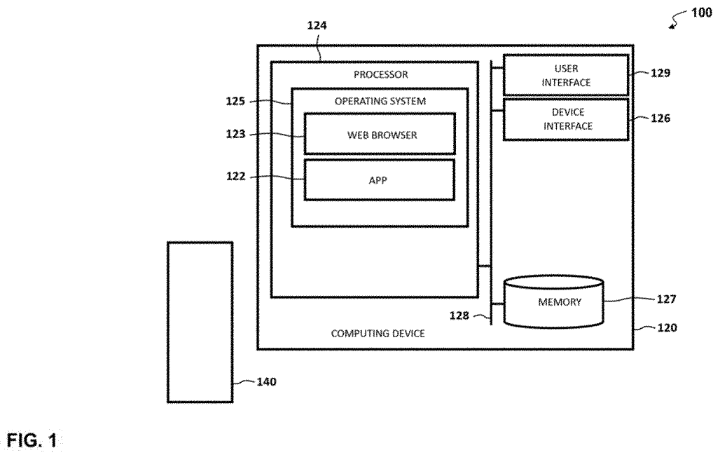

FIGS. 1-19 and the following discussion provide a brief, general description of a suitable computing environment in which aspects of the described technology may be implemented.

Although not required, aspects of the technology may be described herein in the general context of computer-executable instructions, such as routines executed by a general- or special-purpose data processing device (e.g., a server or client computer). Aspects of the technology described herein may be stored or distributed on tangible computer-readable media, including magnetically or optically readable computer discs, hard-wired or preprogrammed chips (e.g., EEPROM semiconductor chips or chip sets), nanotechnology memory, biological memory, or other data storage media. Alternatively, computer-implemented instructions, data structures, screen displays, and other data related to the technology may be distributed over the Internet or over other networks (including wireless networks) on a propagated signal on a propagation medium (e.g., an electromagnetic wave, a sound wave, etc.) over a period of time. In some implementations, the data may be provided on any analog or digital network (e.g., packet-switched, circuit-switched, or other scheme).

The described technology may also be practiced in distributed computing environments where tasks or modules are performed by remote processing devices, which are linked through a communications network, such as a Local Area Network (“LAN”), Wide Area Network (“WAN”), or the Internet. In a distributed computing environment, program modules or subroutines may be located in both local and remote memory storage devices. Those skilled in the relevant art will recognize that portions of the described technology may reside on a server computer, while corresponding portions may reside on a client computer (e.g., PC, mobile computer, tablet, or smartphone). Data structures and transmission of data particular to aspects of the technology are also encompassed within the scope of the described technology.

Present embodiments provide for high-speed, accurate, and versatile prediction of molecular attributes wherein a user may provide three-dimensional data containing the structural information of one or more chosen or user identified molecules and receive a list containing accurate predictions of a wide variety of molecular attributes for each of the provided or user identified molecules. In one embodiment, “Fast, Accurate, and Versatile, In-Silico Measurement” (FAVIM) may execute a Program to receive user inputs containing three-dimensional spatial information of a disease agent receptor target and one or more molecules, prepare an input representation of combined user inputs, and provide said input representation into one or more Transformer Neural Networks (800) to predict one or more attributes of the molecule including, but not limited to, the binding affinity of said molecule with a target receptor. For example, known molecules, and known potential target receptors of disease agents, may be expressed and available for use in Protein Data Bank (PDB) format, OBJ format, or other chemical or three-dimensional file format. Thus, a system user may provide a three-dimensional representation of both a molecule and a target receptor, such as in Protein Data Bank (PDB) format, OBJ format, or other chemical or three-dimensional file format, and the Program may extract the three-dimensional atomic types (i.e., the atomic species such as Oxygen, Hydrogen, Nitrogen, Carbon, etc.) and the atomic coordinates of the atomic types within both the target receptor and the chosen or user selected molecule. If a user provides any molecule or target receptor representations in a format that is not three-dimensional (e.g. Simplified Molecular-Input Line-Entry (SMILE) format), then the system will automatically convert the representations to a three-dimensional representation of the molecule or target receptor, or both, using a third-party software (for example RDKit) before extracting the atomic types and coordinates of the chosen or user provided molecule, target receptor, or both. The System then creates a representation of the three dimensional molecule and the three dimensional target receptor, here vector representations of the three dimensional molecule and target receptor, from the atomic types and coordinates thereof. The three dimensional representations are then embedded and positionally encoded into a full input embedding which is provided to a Transformer Neural Network (800) to predict one or more molecular attributes including, but not limited to, attributes relating to the binding affinity between the chosen or user provided molecule and the target receptor provided by the user.

In one embodiment, the user is able to provide or input to the system the three-dimensional molecule and target receptor representations through a simple user interface which also allows the user to view the molecular attribute predictions on an easy-to-read display. In another embodiment, the software is connected to an alternate, external technology, with which the Program is integrated, from which the software may directly receive the “user inputs” and return the molecular attribute predictions without the use of a graphic interface, in other words, without the need for a graphical user interface. Having both of these options is important for the global battle against disease for different essential uses of the technology. For example, an easy-to-use user interface, which allows artificial intelligence (AI) molecular attribute prediction to be accessible to researchers in any industry, not only limited to software developers, ensures that the pharmacologists and medical experts who need it most are able to harness the power of the technology. Alternatively, those with adequate software expertise can access the inputs and outputs directly and integrate the system provided herein or inputs thereto and outputs therefrom as a key measurement tool within powerful new technologies developed in the future.

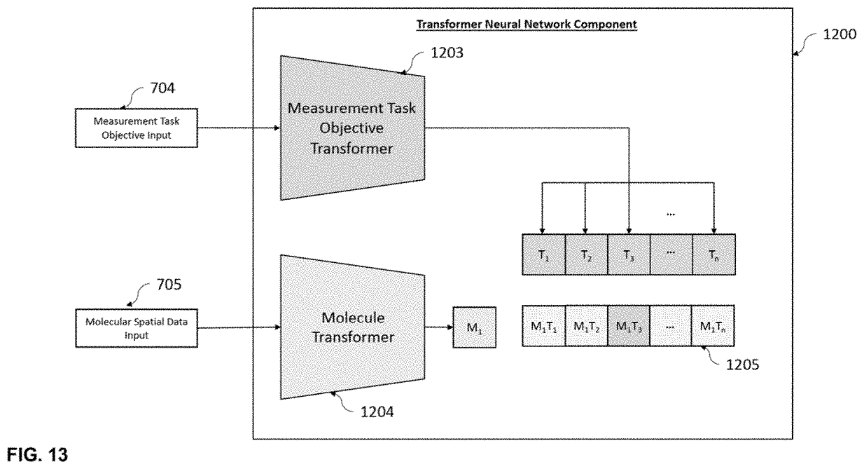

While Transformer Neural Networks (800) are traditionally used for sequential tasks such as natural language processing where strings of data are evaluated, by extrapolating these powerful neural networks into the new domain of a three-dimensional spatial task, the molecular attribute prediction system is able to overcome a variety of limitations seen by previous perceived solutions to determining the likelihood that a molecule can be used to treat an infection caused by an infectious agent. Transformer Neural Networks (800) allow for much more robust calculations of molecular metrics than currently existing virtual screening technologies. The majority of molecular attribute prediction technologies are function-based, meaning they use pre-defined functions to calculate approximate molecular attributes. However, the accurate measurement of binding affinity, or other molecular attributes, requires the computation far too many factors (e.g. atom types, atom charges and polarizations, angles, distance between atoms, rotatable bonds, etc.) for a man-made function to accurately model the full complexity of the chemical attributes of complex molecules. The most advanced molecular attribute prediction technologies currently use three-dimensional Convolutional Neural Networks, but these networks have several inherent limitations which must be improved upon. First, Convolutional Neural Networks are unable to learn sufficiently complex internal representations, for example, the three dimensional structure of atomic species in molecules and the interactivity thereof with other three dimensional atomic structures, such as that of a receptor protein on a virus, etc., because they lack the robust representations created using the Multi-Head Attention Mechanisms (1000) within Transformer Neural Networks (800). In recent research, this has resulted in Transformer Neural Networks (800) out-performing Convolutional Neural Networks on major two-dimensional computer vision benchmarks, so the novel extrapolation of the powerful representations created by Transformer Neural Networks (800) into three-dimensional spatial data allows this advantage to be translated into the domain of molecular chemistry. Another accuracy advantage provided by Transformer Neural Networks is that they are much more scalable than Convolutional Neural Networks, allowing the model to handle far greater complexity and utilize significantly more factors within its computations. Second, Transformer Neural Networks (800) are more efficient than Convolutional Neural Networks, allowing faster and cheaper computation which may allow this life-saving system or methodology to be accessible even to pharmacologists from the world's most impoverished nations. Third, unlike Convolutional Neural Networks, Transformer Neural Networks (800) are capable of Multi-task learning, allowing the same Neural Network used to predict the molecule's Binding Affinity to the target receptor to also predict many other molecular attributes related to the pharma logical applicability thereof, rather than requiring separate Neural Networks for each attribute. Even beyond multi-task learning, in one embodiment, depicted in FIG. 13 and further explained below, the use of a Transformer Neural Network (800) enables zero-shot spatial reasoning in which the neural network is capable of measuring entirely new molecular attributes without any prior training, simply based upon the Transformer Neural Networks' (800) robust internal understandings of both natural language and organic chemistry.

It is understood that while molecules with strong-binding affinity to the target receptor are a good start for discovering a candidate drug, strong-binding affinity is only one of many necessary molecular qualities for effective drugs. There are hundreds, or even thousands, of additional molecular attributes required for a drug candidate to become viable and thereby useful, so the Transformer Neural Network's (800) multi-task and zero-shot capabilities allow much greater prediction of drug candidate efficacy far beyond prior solutions.

For example, Remdisivir® has shown great potential as a candidate drug for COVID-19 throughout the current global pandemic due to its binding affinity to the ACE2 receptor but presents challenges in the production of a sufficient global supply due to the complexity required to synthesize the molecule. Additionally, high-quality drug candidates must not have adverse interactions with other drugs and/or the human or other body, must be able to permeate through any permeation required membranes for absorption thereof into the body, preferably be soluble enough to be orally administered (for patient acceptance) and meet many more requirements. In a robust embodiment depicted in FIG. 13 and further explained below, the capability of zero-shot learning may also enable software capable of inherently understanding and predicting complex molecular properties such as whether a molecule would cause side effects, adverse interactions with other drugs, or potentially even predict performance on specific FDA tests without requiring specialized training or software for the new task.

FIG. 1 illustrates an example of a top-level functional block diagram of a computing system embodiment (100). The example operating environment is shown with a server computer (140) and a computing device (120) comprising a processor (124), such as a central processing unit (CPU) or a graphics processing unit (GPU), addressable memory (127), an external device interface (126), e.g., an optional universal serial bus port and related processing hardware and software, and/or an Ethernet port and related processing hardware and software, and an optional user interface (129), e.g., an array of status lights and one or more toggle switches, and/or a display, and/or a keyboard and/or a pointer-mouse system and/or a touch screen. Optionally, the addressable memory may include any type of computer-readable media that can store data accessible by the computing device (120), such as magnetic hard and floppy disk drives, optical disk drives, magnetic cassettes, tape drives, flash memory cards, digital video disks (DVDs), Bernoulli cartridges, RAMs, ROMs, smart cards, etc. Indeed, any medium for storing or transmitting computer-readable instructions and data may be employed, including a connection port to or node on a network, such as a LAN, WAN, or the Internet. These components or elements may be in communication with one another via a data bus (128).

In some embodiments, via an operating system (125) such as one supporting a web browser (123) and applications (122), the processor (124) may be configured to execute steps of a process establishing a communication channel and processing according to the embodiments described above. In one embodiment, an application (122) is a targeted molecular design application as described below. In another embodiment, the application 122 is an application to determine the likely application of a molecule to treat a specified disease.

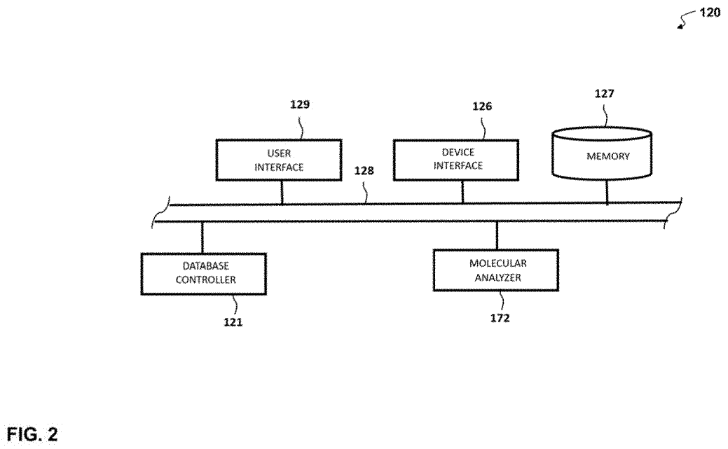

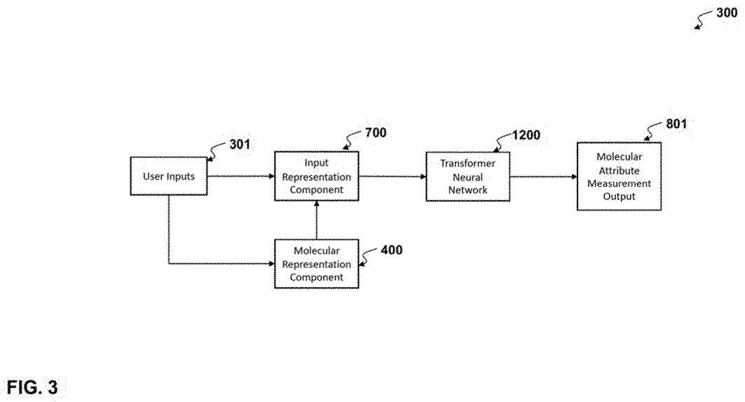

With respect to FIG. 2, components associated with, or in communication with, the processor (124) are shown. A database controller (121) may be in communication with the processor (124), for example, via the data bus (128). In one embodiment, the database controller (121) may receive and store data, such as data from various industries and state agents (e.g., the pharmaceutical industry, the chemical industry, the FDA, etc.) as well as training checkpoints from at least one database, such as a database associated with the server computer (140) in FIG. 1, and load said data into, for example, a cross-platform database program. A user may launch the molecular attribute prediction application (e.g., application 122) to interact with the program at the user interface (129). The application (122) may then access this database at any point to use or store any files used within molecular measurement process, such as the Fast, Accurate, and Versatile, In-silico Measurement (FAVIM) process (300) described in FIG. 3, or in any other processes facilitated by the application (122).

With respect to FIG. 3, a flow diagram depicting the Fast, Accurate, and Versatile, In-silico Measurement process (300) is shown. First, the program receives the User Input (301) from either a User Interface Component (129) or from another technology with which the program is integrated. The User Input (301) may contain, for example, representations of both the target receptor and the molecule to test for the likelihood to bind to the target receptor, which are provided to the Molecular Representation Component (400) for example as a molecule and target receptor pair provided by a user of the system in two dimensional or three dimensional form. The Molecular Representation Component (400) converts both the target receptor and the molecule representation into Numerical Matrix Representations (405) of each, which are then provided to the Input Preparation Component (700). The Input Preparation Component (700) receives the Numerical Matrix Representations (405) of both the target receptor and molecule, and in one embodiment, may also receive a Measurement Task Objective (701) from the User Inputs (301). The measurement task objective scores each attribute or metric for which a molecule is to be evaluated, for example on a one to ten scale, with higher numbers assigned to attributes or metrics which the user has selected as more important than other attributes or metrics. which the user has designated as less important Then, the Input Preparation Component (700) here creates a Numerical Matrix Representation (405) of the Measurement Task Objective (701) and concatenates all of the Numerical Matrix Representations (405) with Start Tokens (703) preceding each respective input type to create a fully prepared Vectorized Input (704, 705). Here, an input type is the language or type of record for, for example, each atom, amino acid, etc. The Vectorized Input (704, 705) is provided to the Transformer Neural Network Component (1200), which will compute a final Molecular Attribute Measurement Output (801). In one embodiment, this Molecular Attribute Measurement Output (801) is provided to the User Interface Component (129) to be displayed to the user. In another embodiment, the Molecular Attribute Measurement Output (801) is provided directly to another technology with which the Program is integrated. In an alternative construct, linguistic 1D representations, such as the amino acid sequence of the protein or the SMILE sequence of the molecule as an input to the molecular representation component 400, and the system learns its own internally embedded understanding of the 3D properties of the molecule without ever creating the explicit 3D coordinates.

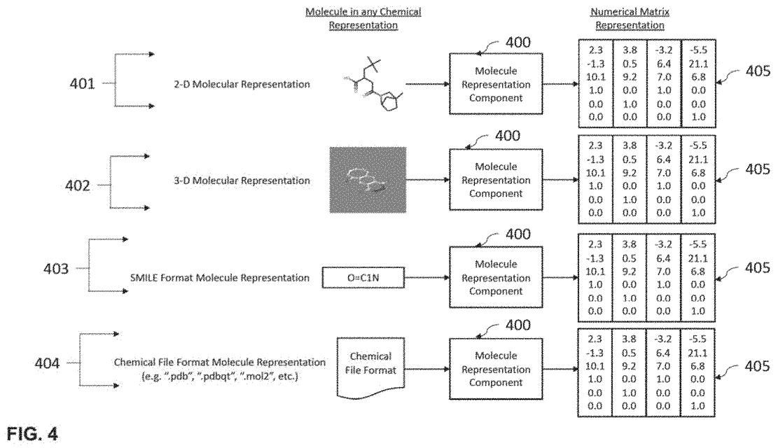

With respect to FIG. 4, a flow diagram of the function of the Molecular Representation Component (400) is shown. Here, the Molecular Representation Component (400) is configured to receive a representation of the molecule and target receptor selected or provided by a user or otherwise provided, in different formats by which the atomic types or atomic species of the molecule and target receptor and their locations within the molecule can be extracted by the Molecular Representation Component (400). For example, the Molecular Representation Component (400) may receive a molecule and/or target receptor in a 2-D representation (401), 3-D representation (402), SMILE Format (403), Chemical File Format (404), or other molecular representation from which the atomic types or species, and their location within the molecule or target receptor, can be identified. From this molecular representation, the Molecular Representation Component (400) extracts both the three-dimensional coordinates and types (e.g. atom types, bond types, location, etc.) to create a Single Molecule Attribute Numeric Representation (500) for each structural attribute (e.g. atoms, bonds, etc.) of the molecule within the molecular representation. In one embodiment, these structural attributes may be automatically extracted from the Chemical File using a third-party software (e.g. RDKit, Open-babel, etc.), using text splitting functions commonly provided automatically by programming languages, or by using custom data extract functions. In another embodiment, input molecular representations may be automatically converted to different molecular representations using a third-party software (e.g. RDKit, Open-babel, etc.) or other molecule format conversion functions. In one embodiment, the Molecular Representation Component (400) is able to take any molecular representation as input and create a sequence of Single Molecular Attribute Numeric Representations (500). Each of these Single Molecular Attribute Numeric Representations (500), i.e., a structural attribute and location thereof, are then concatenated to create a Numerical Matrix Representation (405) which is then provided to the Input Preparation Component (700). In one embodiment, this Numerical Matrix Representation (405) may be converted into a Three-Dimensional Voxel Map (600) within the Molecular Representation Component (400) and be used as the new Numerical Matrix Representation (405) output created by the Molecular Representation Component (400).

In another embodiment, both a ligand (all or part of the molecule) and a target receptor are represented within the same one or more a Numerical Matrix Representation (405) in which the ligand (all or part of the molecule) is represented multiple times in a plurality of one or more poses representing possible positions and configurations in which the ligand (all or part of the molecule) may interact with the target receptor. These one or more poses may be selected using a flooding algorithm, a rotation of the ligand or molecule along the X, Y, and/or Z atomic coordinates' axis, a shift of the relative position of the ligand or molecule along the X, Y, and/or Z atomic coordinates' axis, a pre-determined selection, or another manner of docking pose/configuration identification. In other embodiments, the Numerical Matrix Representations (405) may be formatted differently.

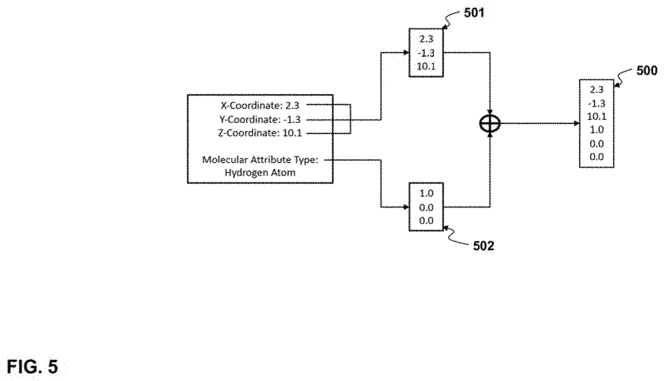

With respect to FIG. 5, a flow diagram of the process to create a Single Molecular Attribute Numeric Representation (500) in one embodiment is shown. For each molecular structural attribute extracted by the Molecular Representation Component (400), the molecular attribute type is one-hot-encoded into a Molecular Attribute Type Vector (502) in which every value is 0 except for the value corresponding to the respective type which is given a value of 1. The X, Y, and Z coordinates are also represented in a Molecular Attribute Coordinates Vector (501) and concatenated with the Molecular Attribute Type Vector (502) to create the full Single Molecular Attribute Numeric Representation (500).

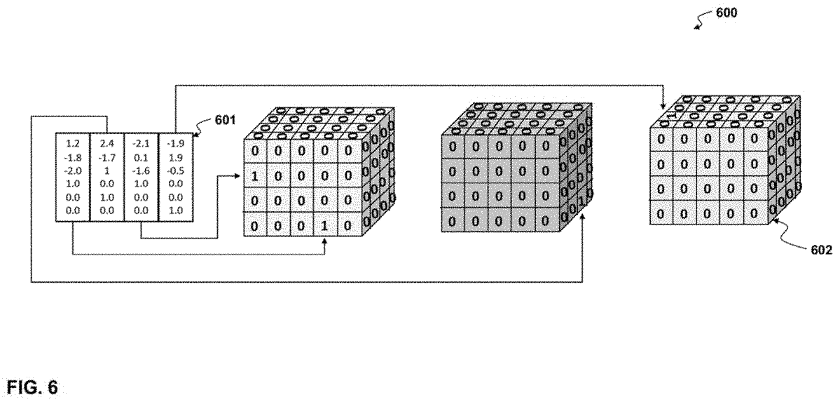

With respect to FIG. 6, a flow diagram for creating a Three-Dimensional Voxel Map (600) within the Molecular Representation Component (400) in one embodiment is shown. In this embodiment, the Numerical Matrix Representation (405) created by the Molecular Representation Component (400) it used to create a Three-Dimensional Voxel Map (600). The Three-Dimensional Voxel Map (600) consists of a three-dimensional binary matrix containing one or more channels, with one channel for each different type of molecule structural attribute. Within each channel, the center of each three-dimensional matrix is considered to be the origin, where the X, Y, and Z atomic coordinates are all equal to 0. In different embodiments, the three-dimensional matrices may have different heights, widths, and depths to reflect the three-dimensional molecular design. First, each of the molecular attribute type's three-dimensional binary matrix channel is initialized to all zeros. Then, for each of the Single Molecular Attribute Numeric Representations (500) representing the structural attributes of the molecule within the Numerical Matrix Representation (405), the location in which the structural attribute's atomic coordinates lie on the is changed from a zero to a one (602) within the molecular attribute type's respective channel matrix, so a one may indicate the presence of that molecular attribute type at the specified location while a zero may indicate the absence of that molecular attribute type at the specified location. Here, the numerical representation includes a three dimensional representation of each attribute, for example an atom and, and a fourth dimension is provided by the attributes location in the voxel map. In one embodiment, both the ligand (molecule) and the receptor are represented within the same Three-Dimensional Voxel Map (600) in which the ligand (molecule) is represented multiple times in a plurality of one or more poses representing positions and configurations in which the ligand (or molecule) may interact with the receptor or other spatial information involving the one or more ligand (or molecules) poses or the one or more receptors. These one or more poses may be selected using a flooding algorithm, a rotation along the X, Y, and/or Z atomic coordinates' axis, a shift along the X, Y, and/or Z atomic coordinates' axis, a pre-determined selection, or another manner of molecular pose/configuration identification. In another embodiment, this the ligand (molecule) may be represented in this plurality of one or more poses separately from the receptor.

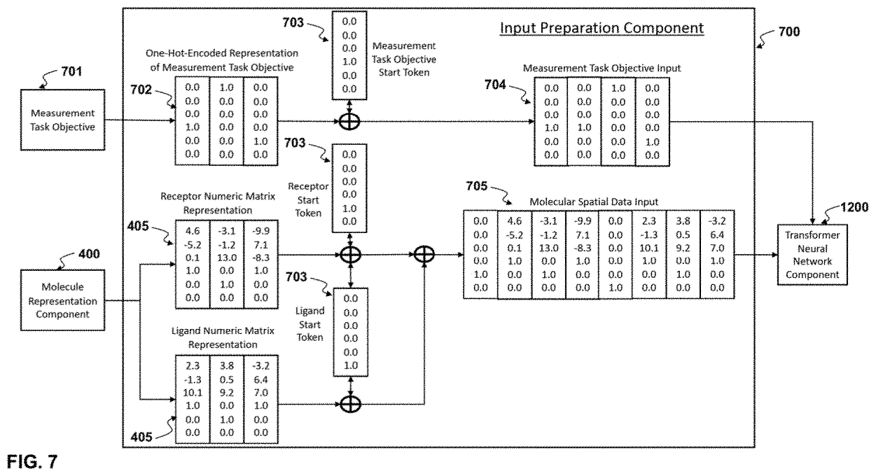

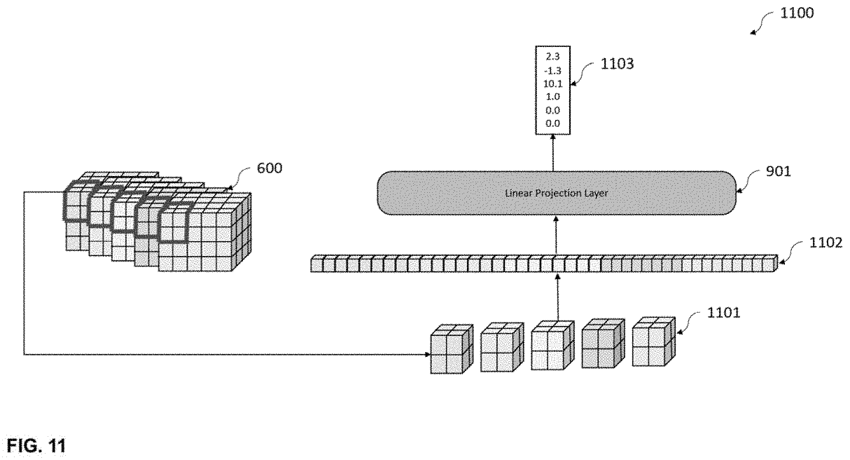

With respect to FIG. 7, a flow diagram of the Input Preparation Component (700) in one embodiment is shown. In one embodiment, the Input Preparation Component (700) receives both the Measurement Task Objective (701) and a Numerical Matrix Representation (405) of one or more ligands (molecules) and a Numerical Matrix Representation (405) of one or more receptors. In another embodiment, no Measurement Task Objective (701) is received. In one embodiment, both the ligand (molecule) and the target receptor are represented within the same one or more Numerical Matrix Representations (406) in which the ligand (molecule) is represented multiple times in a plurality of one or more poses representing positions and configurations in which the ligand (molecule) may interact with the target receptor or other spatial information involving the one or more ligands (molecules) or the one or more receptors. These one or more poses may be selected using a flooding algorithm, a rotation along the X, Y, and/or Z atomic coordinates' axis, a shift along the X, Y, and/or Z atomic coordinates' axis, a pre-determined selection, or another manner of molecular pose/configuration identification. In another embodiment, other numeric matrices and/or vectors may be provided to the Input Representation Component (700). In one embodiment, the Measurement Task Objective (701) is received by the Input Preparation Component (700) in the form of a text string, such as a natural language sentence, and tokenized (split into a list of individual words). Each word is then one-hot-encoded into separate vectors with the length of the vocab size, where the vocab size may be any integer representing the total number of different words which may be used within the Measurement Task Objective (701) and may be different lengths in different embodiments. These one-hot-encoded vectors are binary vectors in which all values are zero except for the value at the index representing the specific word, start token, or other attribute to be represented by the vector, which is set to 1. Each of these one-hot-encoded vectors may be concatenated together into a Binary Numeric Matrix (702), along with a preceding one-hot-encoded vector representing a Measurement Task Objective Start Token (703) to form a numeric Measurement Task Objective Input (704) which is provided to the Transformer Neural Network Component (1200). In other embodiments, this numeric Measurement Task Objective Input (704) may be represented differently. In another embodiment, no numeric Measurement Task Objective Input (704) will be created. In the same manner, any of, or many of, the Numerical Matrix Representation(s) (405) of one or more ligands (molecules) and/or receptors may also be concatenated with preceding start tokens (703) which provide further information regarding each Numerical Matrix Representation (405), such as a Receptor Start Token (703) being concatenated to the beginning of a Receptor's Numerical Matrix Representation (405) and a Ligand (or Molecule) Start Token (703) being concatenated to the beginning of a Ligand's (or Molecule's) Numerical Matrix Representation (405). Each of these concatenated matrix representations may then also be concatenated with each other in order to form a full Molecular Spatial Data Input (705) which is provided to the Transformer Neural Network Component (706). In another embodiment, the Numerical Matrix Representation (405) is in the form of a Three-Dimensional Voxel Map (600), and provided directly to the Transformer Neural Network Component (1200) and the Molecular Spatial Data Input (705) is created within the Transformer Neural Network's Embedding Layer (901) through the process demonstrated in FIG. 11 below. In other embodiments, the Molecular Spatial Data Input (705) may be represented differently.

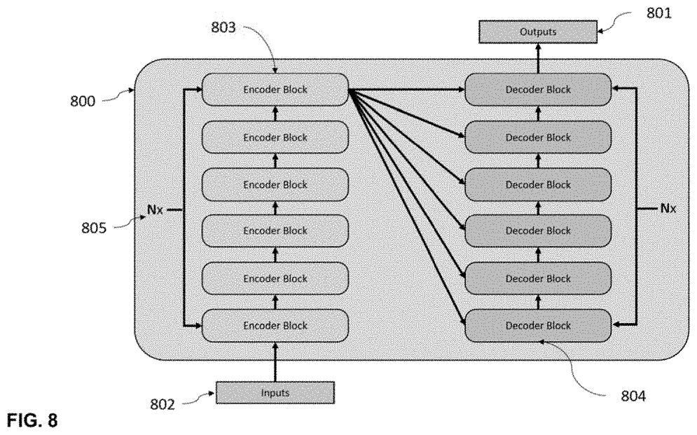

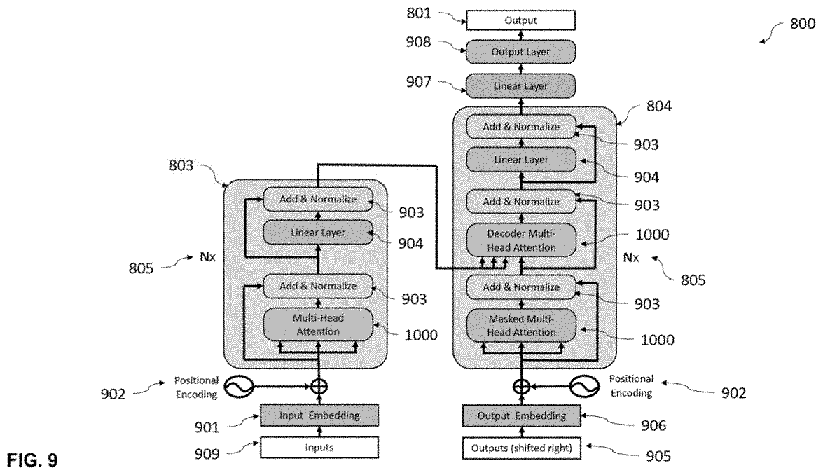

With respect to FIG. 8, a flow diagram of a Transformer Neural Network (800) in one embodiment is shown. In one embodiment, one or more Inputs (802) may be passed to the network and are passed through a series of Nₓ Encoder Blocks (803), where x is a positive integer or 0 and represents the order of each encoder block in the series of Encoder Blocks (803), i.e., encoder block N₀, N₁, N₂, N₃ . . . Nₓ. In different embodiments, or in different Transformer Neural Networks (800) within the same embodiment, the Inputs thereto may vary in length, shape, dimension, number, type, plurality, or other manner of variation. In the same manner, Outputs (2605) of the Transformer Neural Network (800) may vary depending on the Output Layer (908) used within the Transformer Neural Network (800), which is further explained below. In one embodiment, there may be zero, one, two, or even more Encoder Blocks, as demonstrated by “Nx” (805) which demonstrates that this number may be changed to any amount. If the “Nx” (805) for encoders is 0, the Inputs (802) are given directly to a first Decoding Block (804). Where multiple encoder blocks are present, the output of each encoder block, except the final encoder block Nₛ, is given to the next of the encoder block in the series of encoder blocks up to being given to the final encoder block N. The output of the final Encoder Block Nₓ (803) is then given to the first Decoder Block 804, (decoder block N₁) if any decoder block is present and is also given directly from the final encoder block Nₓ to each Decoder Block (804).The output of each decoder block (804) is given to the next decoder block (804) in the series of Decoder Blocks (804). The output of the final Decoder Block Nₓ (804) where one or more are present is passed to the final Linear Layer (907) to compute the Final Output (801) of the Transformer Neural Network (800). Again, here, N decoder blocks, where N is a positive integer or 0, may be employed, and the number represents the order of the decoder block in the series of decoder blocks. If the x in the “Nₓ” (805) for decoders is 0, the output of the final Encoder Block (803) is provided directly to the final Linear Layer (907) (FIG. 9). Otherwise, the final linear layer (907) receives the output from the final Decoder Block Nₓ (804).

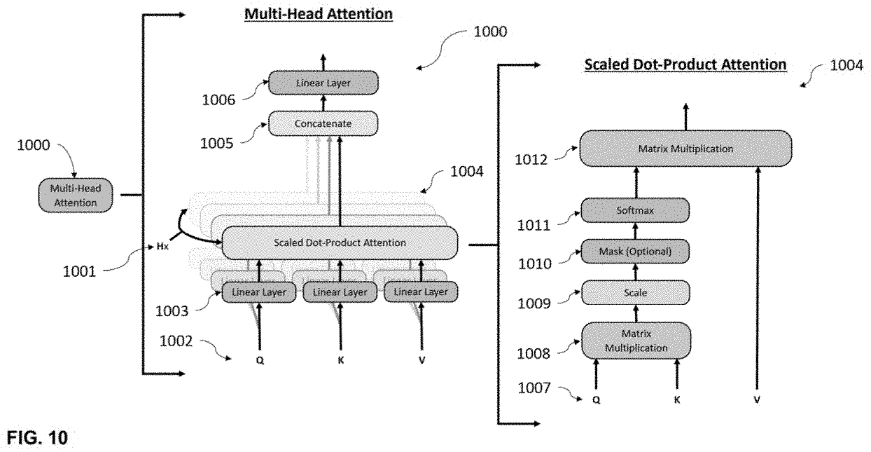

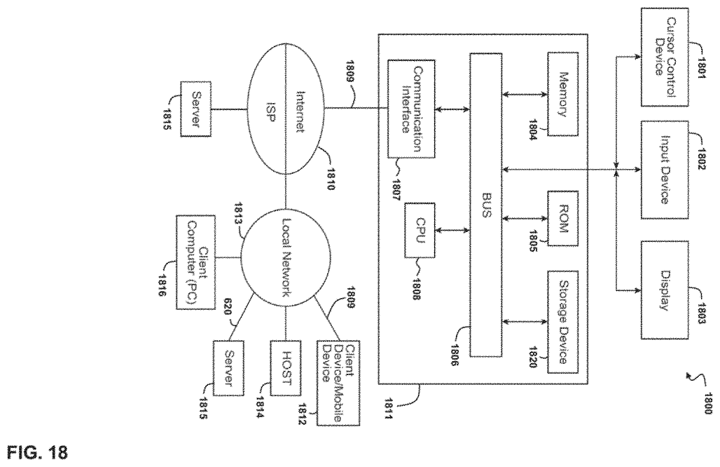

With respect to FIG. 9, a detailed flow diagram of additional componentry of the Transformer Neural Network (800) of FIG. 18 is shown, including that of the Encoder Blocks (803) and Decoder Blocks (804). In one embodiment, the Inputs (802) are embedded in an Input Embedding component (901) to store contextual information, and then positionally encoded in a Positional Encoding component (902), and then these Inputs (802) n are passed to the first Encoder Block (803). Here, positional coding establishes the position of each portion of the molecule (or target receptor). Once embedded and encoded, the inputs in this example are duplicated, where one copy following positional encoding may be given to a first Add & Normalize Layer (903a), and “Hx” (1001) in number of, in the example of FIG. 9 three, copies thereof are given to a Multi-Head Attention Layer (1000a). In ach normalize layer, the input thereto is scaled so that relative values, rather than absolute values, are processed. In one embodiment, a first Add & Normalize Layer (903a) receives both the input copy following positional coding thereof and the output of the Multi-Head Attention Layer (1000a), and the first Add & Normalize Layer (903a) adds the input and output copies together and creates a normalized output which is then sent to both a Linear Layer (904) and another, second Add & Normalize Layer (903b). The Linear Layer (904) transmits its output to the second Add & Normalize Layer (903b), which adds the output with the same input received by the Linear Layer (904), creates a normalized output and sends the output to the next encoder block in the series of encoder blocks, or to the decoder block(s) 804, if the output is from the final encoder block in the series of encoder blocks. Once the inputs have passed through the “Nx” (805) number of encoder blocks, the output of the final encoder block 803 is passed to all of the decoder blocks 804, or directly to the output where no Decoder Blocks (804) are present, and the original Inputs (802) are shifted one time step to the right (905), given new embeddings (906), Positionally Encoded with the positional encoding component (902), and given to the first Decoder Block (804). The inputs to the Decoder Blocks (804) in this example are duplicated, and one input following positional encoding thereof is passed to the third Add & Normalize Layer (903c), and three copies thereof are given to each of the “Hx” (1001) Masked Multi-Head Attention Layers (1000a). Here, the Masked Multi-Head Attention Layer (1000b) has the same structure and operation e as the Multi-Head Attention Layer (1000a) except that it includes the optional Mask (1010) depicted in FIG. 10 below. The third Add & Normalize Layer (903c) receives both the input copy and the output of the Masked Multi-Head Attention Layer (1000b), adds them together, and creates a normalized output which is then sent to the next, or third, Add & Normalize Layer (903c) and to a Decoder Multi-Head Attention Layer (1000c). The Decoder Multi-Head Attention Layer (1000c) also receives the output from the final Encoder Block (803), and then passes the processed output therefrom to the next, or fourth, Add & Normalize Layer (903d), which adds it with the original input received by the Decoder Multi-Head Attention Layer (1000). The fourth Add & Normalize Layer (903d) normalizes the input and output and passes it to both a second Linear Layer (904b) and a fifth Add & Normalize Layer (903e). The final or fifth Add & Normalize Layer (903) sends the output thereof to the next Decoder Block (804), or if it is the final Decoder BlockNₓ (804), the output is provided to one or more Output Linear Layers (907) and then to the final Output Layer (908) to create the Final Output (801) of the Transformer Neural Network (800). The final output 801 may be displayed on a screen, and may, for example be a measure of the likelihood that the molecule will bind to the target receptor. This may, for example, be a numeric output wherein the magnitude of the number is relative to the likelihood that the molecule will bind to the target receptor.

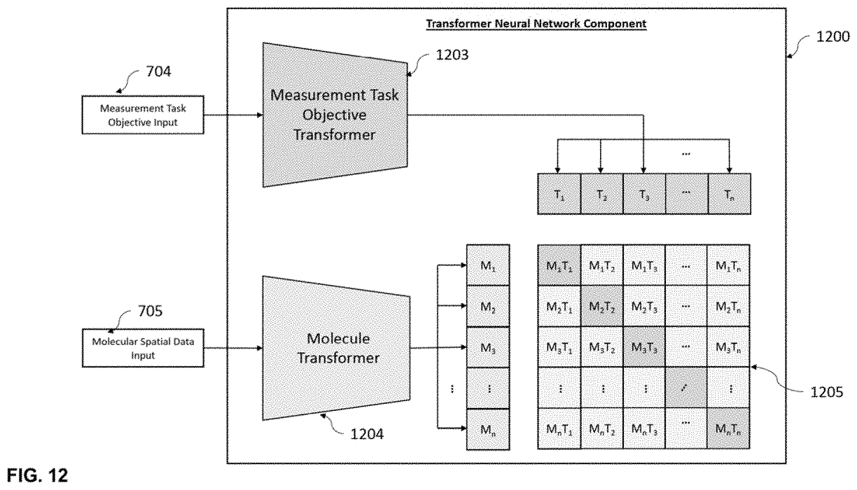

This technology may offer humanity a robust lifeline in a time of great need through the enablement of state-of-the-art, in-silico drug screening, which is able to alleviate barriers to drug discovery and drastically reduce both the time and cost required to combat disease. With such a large potential to benefit society, it is essential that the benefits of this technology be accessible to all, offering protection from the many deadly pathogens, regardless of world-wide location or economic status. Unfortunately, researchers and pharmacologists within impoverished nations or low-income locations may have inadequate resources to overcome the computational burden required by highly sophisticated, artificially intelligent technology. This problem may be alleviated through the use of different final Output Layers (908) within different embodiments of the software. While a large, robust embodiment of this software, such as the Contrastive Language-In-Silico Pre-training (CLISP) design, depicted in FIGS. 12 and 13, in its Transformer Neural Network Component (1200) may provide many revolutionary opportunities for diverse, generalizable drug screening through its profound multi-task and zero-shot learning capabilities (which are further explained below), such a large Transformer Neural Network Component (1200) may require the use of hundreds or even thousands of powerful computers, costing thousands or millions of dollars and preventing the technology from being accessible to anyone other than the wealthy. However, even without full natural-language-capable, zero-shot in-silico drug screening, this technology may still revolutionize impoverished nations' ability to combat disease. For example, in one embodiment, the transformer Neural Network Component (1200) consists of a single Transformer Neural Network (800), unlike the CLISP design which requires two, and contains a single output unit with a Sigmoid

$$( \frac{1}{1 \pm x} )$$

activation function in the Output Layer (908) and the Transformer Neural Network (800) is trained using a binary cross entropy loss. This activation function results in a single binary classification output, such as whether the ligand (molecule) would effectively inhibit the target receptor or not. While this forgoes the multi-task learning capabilities offered by Transformer Neural Networks (800) which allow the determination of multiple properties, in addition to whether it will effectively inhibit a target receptor by binding thereto, it allows a much smaller Transformer Neural Network (800) to be used (e.g. decreasing hyper-parameters such as number of encoder/decoder layers, number of attention heads, embedding dimensions, or other size-related hyper-parameters common in Transformer Neural Networks (800)) while maintaining high accuracy due to the other performance enhancements provided by Transformer Neural Networks (800).

A common metric for comparison of the performance capabilities of Neural Networks is the number of trainable parameters within the network. For comparison, the top state-of-the-art neural network currently used for this type of binary classification measurement of binding affinity is a standard three-dimensional convolutional neural network and contains roughly 300 million trainable parameters, requiring the use of ten graphics processing units (GPUs). However, with the enhanced computational efficiency provided by Transformer Neural Networks (800) (along with additional algorithmic improvements further explained below), the Transformer Neural Network Component (1200) may be scaled to contain up to 1.6 billion parameters even on a single graphics processing unit (GPU), providing a technology that is five times more powerful at one-tenth of the computational cost than the previously state-of-the art solution. Therefore, state-of-the-art performance may still be achieved with only a single computer through this embodiment, so those lacking computational resources may simply be provided training-weight files, containing the model's pre-trained parameter values, for each molecular measurement which may be loaded into the Transformer Neural Network Component (1200) to measure each respective metric one-at-a-time. Alternatively, in another embodiment, a token representing the desired molecular metric for which to measure may be concatenated with the Molecular Spatial Data Input (705) in a similar manner to the previously mentioned start tokens in order to still harness some of the multi-task learning capabilities provided by Transformer Neural Networks (800).

In yet another embodiment, the Output Layer (908) may contain a single output unit with no activation function in order to provide numeric regression outputs (such as the exact value for the Binding Affinity IC50, Log P solubility, Molecular Weight, etc.). In yet another embodiment a SoftMax function, which predicts a probability distribution across all available classes, may be used for classification tasks, prediction of the next word/value (similar to a common chat-bot used in in natural language processing), and for the Molecule Transformer in the Contrastive Language-In-Silico Pre-training (CLISP) design depicted in FIGS. 12 and 13. In another embodiment, multiple of these output units/activation functions may be used. In other embodiments, other Output Layers (908) may be used.

With respect to FIG. 10, a flow diagram of a Multi-Head Attention Layer component (1000) in one embodiment is shown on the left panel, and a corresponding flow diagram of the operation of a Scaled-Dot Product Attention Component (1004) thereof in one embodiment is shown on the right panel.

In this embodiment, a Multi-Head Attention Layer Component (1000) receives three copies (1002) of inputs (802) for each “Hx” (1001) of the number of attention heads thereof, which copies are each passed through their own respective Linear Layers (1003) dedicated to specific ones of the attention heads thereof, and then given to the respective Scaled Dot-Product Attention Heads (1004). The outputs from all of the Scaled Dot-Product Attention Heads (1004) are concatenated (1005), and passed through another Linear Layer (1006) to create the final output of the Multi-Head Attention Layer (1000). On the right side of FIG. 10, a detailed flow diagram of the functions within the Scaled Dot-Product Attention Head Component (1004) is shown. Each of the Scaled Dot-Product Attention Heads (1004) here receives the three Input copies (1007) from the previous respective Linear Layers (1003) and performs Matrix Multiplication (1008) on two of the three Input copies (1007) to create a new matrix, and scales the new matrix with a Scale Component (1009). The scaling is performed by dividing the new matrix by the square root of the dimension of the Input Copies (1007). A Mask (1010) may optionally be applied next to make the layer a Masked Multi-Head Attention Layer (1000), which provides for zeroing out numbers above the matrix diagonal. Next, a Softmax function is performed with a Softmax Layer (1011), and then sent alongside a remaining copy (1007) to another, second, Matrix Multiplication Layer (1008b) which performs the same operations on the remaining copy and the output of the softmax layer 1011 as was performed on the copies Q and K in the first matrix Multiplication layer 1008, to create the final Scaled-Dot Product Attention output (1004).

With respect to FIG. 11, a diagram depicting an input embedding in one embodiment is shown. Similar to a Vision Transformer, a Three-Dimensional Voxel Map (600) is segmented into patches, which are then flattened into a Flatted Patch Vector (1102) and passed through a Linear Layer to create an Embedded Input Vector (1103). The entire voxel map is patched., and each patch flattened into an embedded input vector 1103, the patches sequenced in the order thereof in the three dimensional voxel map 600. This process is performed simultaneously for each separate patch, and all of the Embedded Input Vectors (1103) are then concatenated to form the full Input Embedding (901) to be later Positionally Encoded (902) and given to the first encoder block of one or more encoder blocks 803, or where no encoder block is used, to a first decoder block of one or more decoder blocks 804. In another embodiment, the Molecular Spatial Data Input (705) is in the form of a Numeric Matrix Representation rather than the Three-Dimensional Voxel Map (600), so the patch-flattening step is skipped, and each token or Single Molecular Attribute Vector (500) within the Molecular Spatial Data Input (705) is linearly projected instead of the Flatted Patch Vector (1102).

With respect to FIG. 12, a diagram depicting a Contrastive Language-In-Silico Pre-training (CLISP) training process within a Transformer Neural Network Component (1200) in one embodiment is shown. Within the Transformer Neural Network Component (1200), there are two Transformer Neural Networks (800): the Measurement Task Objective Transformer (1203) which receives the Measurement Task Objective Input (704), and the Molecule Transformer (1204) which receives the Molecular Spatial Data Input (705). During Pre-training, the Molecular Spatial Data Input (705) is created using true examples of molecules and associated target receptors from known databases which may be used as training data, and the Measurement Task Objective Input (704) is constructed using molecular annotations describing molecular metrics, measurements, and other info regarding the molecule, receptor, and their interactions. The number of molecules used for pre-training is a trade off between the need to train the system and the computing power needed to process a large number of molecules. Both of these Transformer Neural Networks (800) may use a linear projection with any equal number of two or more units for their Output Layers (908). Both of these Transformer Neural Networks (800) may be trained by providing the Molecule Transformer (1204) with true Molecular Spatial Data Inputs (705) and providing the Measurement Task Objective Transformer (1203) with corresponding Measurement Task Objective Inputs (704) created using molecular annotations, some of which are correctly paired annotations and some of which are falsely paired annotations. Both Transformer Neural Networks (800) may then create latent-space output embeddings to represent their respective input, and the models are both trained to maximize the cosine similarity of their latent-space embedding representations of the correctly paired annotations (depicted in FIG. 12 as blue) and minimize the cosine similarity of their latent-space embedding representations of the falsely paired annotations (depicted in FIG. 12 as gray). A simplified explanation of what this process is doing can be thought of as Molecule Transformer (1204) being trained to gain a deep, complex internal understanding of the chemical and pharmaceutical intricacies of the Molecular Spatial Data Input (705), and the other Measurement Task Objective Transformer (1203) is trained to act as a translator by gaining a robust, complex internal understanding of the language in which the molecular annotations were written and learning to correlate the semantic meanings of the annotations to the corresponding latent-space embeddings created by the Molecule Transformer (1204). In another embodiment, an alternative training process may be used.

With respect to FIG. 13, a diagram depicting the training of a Transformer Neural Network Component (1200) utilizing a Contrastive Language-In-Silico Pre-training (CLISP) design is shown. The Molecule Transformer (1204) receives Molecular Spatial Data Inputs (705) and outputs the same latent-space embeddings as described above. At the same time, the Measurement Task Objective Transformer (1203) receives multiple Measurement Task Objective Inputs (704) containing separate numeric input matrices describing two or more classes between which to classify, such as two natural language sentences with one stating that the ligand (or molecule) inhibits (binds with) the receptor and another saying that the ligand (or molecule) does not bind with the receptor. A SoftMax activation function may then be applied to the cosine-similarities of the latent-space embedding created by the Measurement Task Objective Transformer (1203) for each class with the latent-space embedding created by the Molecule Transformer (1204) to calculate a probability distribution amount all of the classes. This enables Zero-Shot classification as shown in FIG. 13, meaning that the Transformer Neural Network Component (1200) may be capable of accurately classifying new molecular metrics which it has never been specifically trained for because the Molecule Transformer (1204) learns to gain an extremely deep and robust internal understand of chemistry and pharmacology that it may predict complex inter-relationships between molecular attributes unknown to humans while the Measurement Task Objective Transformer (1203) gains an equally robust understanding of the semantic interpretations within natural language and how to translate those semantics to numeric latent-space embeddings created by the Molecule Transformer (1204).As a result, when encountering a non-known molecule and target receptor pair, the best fit of the molecule to a receptor will be identified as shown in FIG. 13.

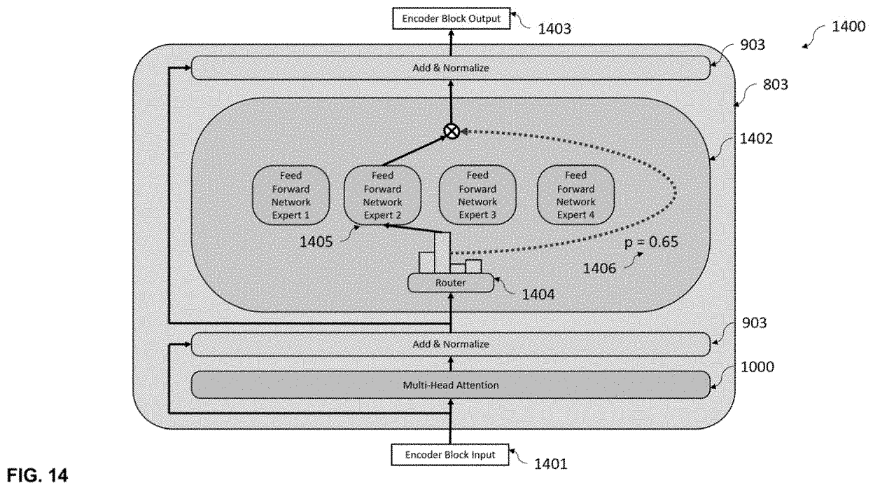

With respect to FIG. 14 a flow diagram, depicting an alternative Switch Transformer Encoder Block (1400) in one embodiment is depicted. In one embodiment, the present flow diagram process accomplishes similar tasks as the previously described Encoder Blocks (803). The Input (1401) received by the Encoder Block (803a) is passed through a Multi-Head LSH Attention Layer (1000) and an Add & Normalize Layer (903) in the same manner and as described with respect to FIG. 9, but then rather than being passed therefrom to a Linear Layer (904), this output is instead passed to a Switch Gate Layer (1402). The input to the Switch Gate Layer (1402) is first received by a Router (1404) which uses trained parameters in the same manner as a Linear Layer (904), which are then normalized using a SoftMax activation function over all of a number of available Feed Forward Network Experts (1405). This creates a probability which determines which expert to route the input received by the Switch Gate Layer (1402) to. The Feed Forward Network Experts (1405) with the largest probability determined by the Router (1404) receives the input received by the Switch Gate Layer (1402), performs feed forward network computation (a similar computation to the use of one or more Linear Layers) on this input received by the Switch Gate Layer (1402), and then the output from that computation is multiplied with the probability determined by the Router (1404), and provided to the Add & Normalize Layer (903) as the final output of the Switch Gate Layer (1402). The output of the Add & Normalize Layer (903) is then used as the final Encoder Block (804) output. These same concepts of Switch Gate Layers (1402) can be applied to Decoder Blocks in the same way and provide a significantly computationally efficient implementation of the Transformer Neural Network Component (1200) in some scenarios.

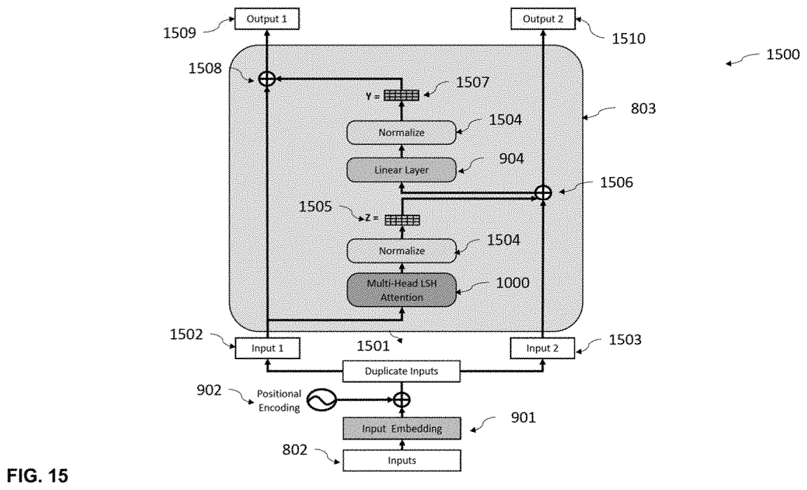

With respect to FIG. 15, a flow diagram of an Encoder Block (803) with Reversible Residual Layers (1500) and Locality-Sensitive-Hashing (1000) is shown. In one embodiment, the present flow diagram process accomplishes similar tasks as the previous Encoder Blocks (803). In one embodiment, Inputs (802) are embedded in an embedding component (902) to store contextual information, and are then positionally encoded in a Positionally Encoded component (901), and then passed to the first Encoder Block (803). The Inputs (802) may be duplicated by a duplicator component (1501) into an Input 1 (1502) and an Input 2 (1503) and used for two identical copies of the model. In the first model copy, Input 1 (1502) is passed to a Multi-Head LSH Attention Layer (1000). The term LSH stands for “locality-sensitive hashing”, which is very similar to the previous Multi-Head Attention Layers (1000) except that the Multi-Head LSH Attention Layer (1000) uses locality-sensitive hashing (LSH) rather than full dot-product Matrix Multiplication (1009). The Multi-Head LSH Attention Layer (1000) output is then passed to a Normalization Layer (1504) to create an Output Z (1505). The Output Z (1505) may then be used as an Output 2 (1510) which is one of the two model outputs, but Output 2 (1510) is added to Input 2 (1503) then passed to a Linear Layer (904). The Linear Layer (904) passes its output to another Normalization Layer (1504) to create an Output Y (1507), which is added to a copy of Input 1 (1502) and used as Output 1 (1509), which is the other model output. Splitting the Add & Normalization Layers (1504) into computing the addition section separately in different model copies allows activations to be recalculated during backpropagation so that the different model copies do not have to be stored, dramatically reducing memory requirements. These same concepts of LSH and Reversible Residual Layers can be applied to Decoder Blocks in the same way and provide a significantly computationally efficient implementation of the Transformer Neural Network Component (1200) in some scenarios.

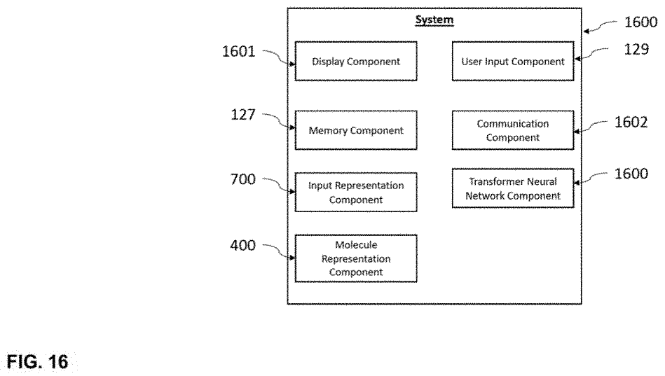

With respect to FIG. 16, a block diagram of an additional system (1600) for Applying Transformer Neural Networks (800) to Spatial Data is shown. The system (1600) may include a Display Component (1601), a User Input Component (129), a Memory Component (127), a Communication Component (1602), an Input Preparation Component (700), a Transformer Neural Network Component (1200), and a Molecule Representation Component (400). In one embodiment, the Display Component (1601) displays the User Interface on the System (1600), which the user may interact with using the User Input Component (129). In one embodiment, the User Input Component (129) may consist of a keyboard and/or mouse, a touchscreen in another embodiment, or other input devices in other embodiments. The Memory Component (127) may contain protein receptor files, databases of molecules, previous experiment history, and other past files uploaded by the user. Here, in contrast to the system of FIGS. 1 and 2, this system is specifically configured to access modules remote thereto in a distributed computing environment.

The Communication Component (1602) may be configured to establish a connection between the System (1600) and any number of external molecule databases in order to send and/or retrieve additional molecule data for the Memory Component (127).

The Transformer Neural Networks Component (1200) may consist of one or many Transformer Neural Networks (800), or other similar Neural Network containing a Multi-Head Attention Mechanism (1000), which may be used to create the Molecular Attribute Measurement Output (801), The Transformer Neural Network Component (1200) may be configured to assign measurements and/or other forms of scores to molecules, for a large variety of molecular attributes. The Molecule Representation Component (400) may be configured to convert the representations of molecules and/or protein receptors between different molecular representation including but not limited to SMILE format representation, binary array representation, 3-D structural graph representation, three-dimensional voxel map representation, and any other molecular representation format needed by other components within the System (1600). The Input Preparation Component (700) may be configured to convert representations of molecules and/or protein receptors from any format received from the Molecule Representation Component (400) into any representation usable by the Transformer Neural Network Component (1200) including, but not limited to, a Three-Dimensional Voxel Map (600), a numerical matrix of molecular attribute types and atomic coordinates such as depicted in the Molecular Spatial Data Input (705) in FIG. 7, or other numeric format which is usable for the Transformer Neural Network Component (1200). Additionally, the Input Preparation Component (700) may be configured to receive representations of Measurement Task Objective (701) in any format including, but not limited to, natural language text, key words/tokens, numerical representations, or other format to represent a Measurement Task Objective (701) and convert them into a Measurement Task Objective Input (704) either in the format depicted in FIG. 7 or in any other numeric format which is usable for the Transformer Neural Network Component (1200).

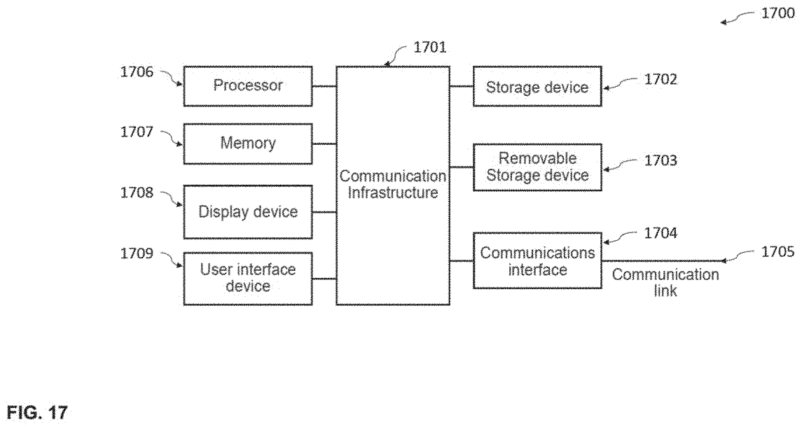

FIG. 17 is a high-level block diagram showing another additional architecture of a computing system comprising a computer system useful for implementing an embodiment of the system and process, disclosed herein. Embodiments of the system may be implemented in different computing environments. The computer system includes one or more processors (1706), and can further include an electronic display device (1708) (e.g., for displaying graphics, text, and other data), a main memory (1707) (e.g., random access memory (RAM)), storage device (1820), a removable storage device (1703) (e.g., removable storage drive, Graphics Processing Unit (GPU), a removable memory module, a magnetic tape drive, an optical disk drive, a computer readable medium having stored therein computer software and/or data), User Interface Device (1709) (e.g., keyboard, touch screen, keypad, pointing device), and a communication interface (1704) (e.g., modem, a network interface (such as an Ethernet card), a communications port, or a PCMCIA slot and card). The Communication Interface (1704) allows software and data to be transferred between the computer system and external devices. The system further includes a communications infrastructure (1701) (e.g., a communications bus, cross-over bar, or network) to which the aforementioned devices/modules are connected as shown.

Information transferred via communications interface (1704) may be in the form of signals such as electronic, electromagnetic, optical, or other signals capable of being received by communications interface (1704), via a communication link (1705) that carries signals and may be implemented using wire or cable, fiber optics, a phone line, a cellular/mobile phone link, a radio frequency (RF) link, and/or other communication channels. Computer program instructions representing the block diagram and/or flowcharts herein may be loaded onto a computer, programmable data processing apparatus, or processing devices to cause a series of operations performed thereon to produce a computer-implemented process.

Embodiments have been described with reference to flowchart illustrations and/or block diagrams of methods, apparatus (systems) and computer program products according to embodiments. Each block of such illustrations/diagrams, or combinations thereof, can be implemented by computer program instructions. The computer program instructions when provided to a processor produce a machine, such that the instructions, which execute via the processor, create means for implementing the functions/operations specified in the flowchart and/or block diagram. Each block in the flowchart/block diagrams may represent a hardware and/or software module or logic, implementing embodiments. In alternative implementations, the functions noted in the blocks may occur out of the order noted in the figures, concurrently, etc.

Computer programs (i.e., computer control logic) are stored in main memory and/or secondary memory. Computer programs may also be received via a communications interface (1704). Such computer programs, when executed, enable the computer system to perform the features of the embodiments as discussed herein. In particular, the computer programs, when executed, enable the processor and/or multi-core processor to perform the features of the computer system. Such computer programs represent controllers of the computer system.

FIG. 18 shows a block diagram of an example system (1800) in which an embodiment may be implemented. The system (1800) includes one or more client devices (1812) such as consumer electronics devices, connected to one or more server computing systems (1815). A server (1815) includes a bus (1804) or other communication mechanism for communicating information, and a processor (CPU and/or GPU) (1808) coupled with the bus (1806) for processing information. The server (1815) also includes a main memory (1804), such as a random-access memory (RAM) or other dynamic storage device, coupled to the bus (1806) for storing information and instructions to be executed by the processor (1808). The main memory (1804) also may be used for storing temporary variables or other intermediate information during execution or instructions to be executed by the processor (1808). The server computer system (1815) further includes a read only memory (ROM) (1805) or other static storage device coupled to the bus (1806) for storing static information and instructions for the processor (2404). A storage device (1820), such as a magnetic disk or optical disk, is provided and coupled to the bus (1806) for storing information and instructions. The bus (1806) may contain, for example, thirty-two address lines for addressing video memory or main memory (1804). The bus (1806) can also include, for example, a 32-bit data bus for transferring data between and among the components, such as the CPU (1808), the main memory (1804), video memory and the storage (1820).

Alternatively, multiplex data/address lines may be used instead of separate data and address lines.

The server (1815) may be coupled via the bus (1806) to a display (1803) for displaying information to a computer user. An input device (1802), including alphanumeric and other keys, is coupled to the bus (1806) for communicating information and command selections to the processor (1808). Another type or user input device comprises cursor control (1801), such as a mouse, a trackball, or cursor direction keys for communicating direction information and command selections to the processor (1808) and for controlling cursor movement on the display (1803).

According to one embodiment, the functions are performed by the processor (1808) executing one or more sequences of one or more instructions contained in the main memory (1804). Such instructions may be read into the main memory (1804) from another computer-readable medium, such as the storage device (1820). Execution of the sequences of instructions contained in the main memory (1804) causes the processor (1808) to perform the process steps described herein. One or more processors in a multi-processing arrangement may also be employed to execute the sequences of instructions contained in the main memory (1804). In alternative embodiments, hard-wired circuitry may be used in place of or in combination with software instructions to implement the embodiments. Thus, embodiments are not limited to any specific combination of hardware circuitry and software.

The terms “computer program medium,” “computer usable medium,” “computer readable medium”, and “computer program product,” are used to generally refer to media such as main memory, secondary memory, removable storage drive, a hard disk installed in hard disk drive, and signals. These computer program products are means for providing software to the computer system. The computer readable medium allows the computer system to read data, instructions, messages or message packets, and other computer readable information from the computer readable medium. The computer readable medium, for example, may include non-volatile memory, such as a floppy disk, ROM, flash memory, disk drive memory, a CD-ROM, and other permanent storage. It is useful, for example, for transporting information, such as data and computer instructions, between computer systems. Furthermore, the computer readable medium may comprise computer readable information in a transitory state medium such as a network link and/or a network interface, including a wired network or a wireless network that allow a computer to read such computer readable information. Computer programs (also called computer control logic) are stored in main memory and/or secondary memory. Computer programs may also be received via a communications interface. Such computer programs, when executed, enable the computer system to perform the features of the embodiments as discussed herein. In particular, the computer programs, when executed, enable the processor multi-core processor to perform the features of the computer system. Accordingly, such computer programs represent controllers of the computer system.

Generally, the term “computer-readable medium” as used herein refers to any medium that participated in providing instructions to the processor (1808) for execution. Such a medium may take many forms, including but not limited to, non-volatile media, volatile media, and transmission media. Non-volatile media includes, for example, optical or magnetic disks, such as the storage device (1820). Volatile media includes dynamic memory, such as the main memory (1804). Transmission media includes coaxial cables, copper wire and fiber optics, including the wires that comprise the bus (1806). Transmission media can also take the form of acoustic or light waves, such as those generated during radio wave and infrared data communications.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, an EPROM, a FLASH-EPROM, any other memory chip or cartridge, a carrier wave as described hereinafter, or any other medium from which a computer can read.

Various forms of computer readable media may be involved in carrying one or more sequences of one or more instructions to the processor (1808) for execution. For example, the instructions may initially be carried on a magnetic disk of a remote computer. The remote computer can load the instructions into its dynamic memory and send the instructions over a telephone line using a modem. A modem local to the server (1815) can receive the data on the telephone line and use an infrared transmitter to convert the data to an infrared signal. An infrared detector coupled to the bus (1806) can receive the data carried in the infrared signal and place the data on the bus (1806). The bus (1806) carries the data to the main memory (1804), from which the processor (1808) retrieves and executes the instructions. The instructions received from the main memory (1804) may optionally be stored on the storage device (1820) either before or after execution by the processor (1808).

The server (1815) also includes a communication interface (1807) coupled to the bus (1806). The communication interface (1807) provides a two-way data communication coupling to a network link (1809) that is connected to the worldwide packet data communication network now commonly referred to as the Internet (1810). The Internet (1810) uses electrical, electromagnetic or optical signals that carry digital data streams. The signals through the various networks and the signals on the network link (1809) and through the communication interface (1807), which carry the digital data to and from the server (1815), are exemplary forms or carrier waves transporting the information.

In another embodiment of the server (1815), interface (1807) is connected to a network (1813) via a communication link (1809). For example, the communication interface (1807) may be an integrated services digital network (ISDN) card or a modem to provide a data communication connection to a corresponding type of telephone line, which can comprise part of the network link (1809). As another example, the communication interface (1807) may be a local area network (LAN) card to provide a data communication connection to a compatible LAN. Wireless links may also be implemented. In any such implementation, the communication interface (1807) sends and receives electrical electromagnetic or optical signals that carry digital data streams representing various types of information.

The network link (1809) typically provides data communication through one or more networks to other data devices. For example, the network link (1809) may provide a connection through the local network (1813) to a host computer (1814) or to data equipment operated by an Internet Service Provider (ISP). The ISP in turn provides data communication services through the Internet (1810). The local network (1813) and the Internet (1810) both use electrical, electromagnetic or optical signals that carry digital data streams. The signals through the various networks and the signals on the network link (1809) and through the communication interface (1807), which carry the digital data to and from the server (1815), are exemplary forms or carrier waves transporting the information.

The server (1815) can send/receive messages and data, including e-mail, program code, through the network, the network link (1809) and the communication interface (1807). Further, the communication interface (1807) can comprise a USB/Tuner and the network link (1809) may be an antenna or cable for connecting the server (1815) to a cable provider, satellite provider or other terrestrial transmission system for receiving messages, data and program code from another source.

The example versions of the embodiments described herein may be implemented as logical operations in a distributed processing system such as the system (1800) including the servers (1815). The logical operations of the embodiments may be implemented as a sequence of steps executing in the server (1815), and as interconnected machine modules within the system (1800). The implementation is a matter of choice and can depend on performance of the system (1800) implementing the embodiments. As such, the logical operations constituting said example versions of the embodiments are referred to for e.g., as operations, steps or modules.

Similar to a server (1815) described above, a client device (1812) can include a processor, memory, storage device, display, input device and communication interface (e.g., e-mail interface) for connecting the client device to the Internet (1810), the ISP, or LAN (1813), for communication with the servers (1815).

The system (1800) can further include computers (e.g., personal computers, computing nodes) (1816) operating in the same manner as client devices (1809), where a user can utilize one or more computers (1816) to manage data in the server (1815).

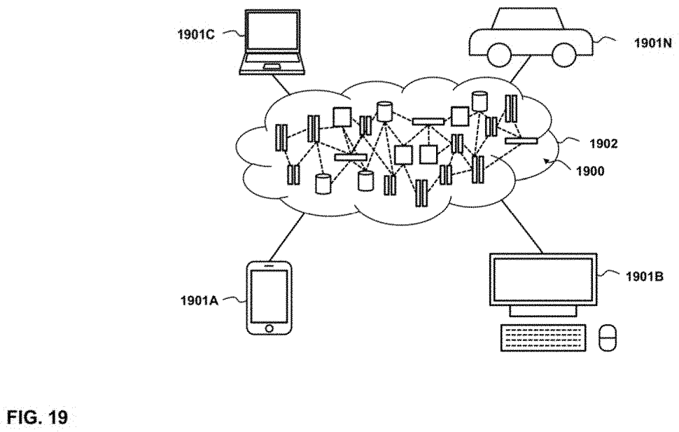

Referring now to FIG. 19, illustrative cloud computing environment (1900) is depicted. As shown, cloud computing environment (1900) comprises one or more cloud computing nodes (1902) with which local computing devices used by cloud consumers, such as, for example, personal digital assistant (PDA), smartphone, smart watch, set-top box, video game system, tablet, mobile computing device, or cellular telephone (1901A), desktop computer (1901B), laptop computer (1901C), and/or automobile computer system (1901N) may communicate. Nodes (1902) may communicate with one another. They may be grouped (not shown) physically or virtually, in one or more networks, such as Private, Community, Public, or Hybrid clouds as described hereinabove, or a combination thereof. This allows cloud computing environment (1900) to offer infrastructure, platforms and/or software as services for which a cloud consumer does not need to maintain resources on a local computing device. It is understood that the types of computing devices 1901A-N shown in FIG. 19 are intended to be illustrative only and that computing nodes (1902) and cloud computing environment (1900) can communicate with any type of computerized device over any type of network and/or network addressable connection (e.g., using a web browser).

It is contemplated that various combinations and/or sub-combinations of the specific features and aspects of the above embodiments may be made and still fall within the scope of the invention. Accordingly, it should be understood that various features and aspects of the disclosed embodiments may be combined with or substituted for one another in order to form varying modes of the disclosed invention. Further, it is intended that the scope of the present invention is herein disclosed by way of examples and should not be limited by the particular disclosed embodiments described above.

What is claimed is:

1. A method of determining the applicability of a candidate molecule for the treatment of an infectious disease, wherein the infectious disease is caused by a multi-atom infectious agent, comprising:
identifying the spatial data of the candidate molecule in three dimensions;
identifying the special data of the infectious agent;
determining the likelihood that the candidate molecule would bind to the infectious agent; and
identifying the suitability of the candidate molecule for a pharmaceutical application based upon at least one candidate molecule property in addition to the likelihood that the candidate molecule would bind to the infectious agent.

2. The method of claim 1, wherein the infectious agent is a target receptor and the likelihood that the target molecule would bind to the infectious agent is the likelihood that the target molecule would bind to the target receptor.

3. The method of clam 2, wherein the step of determining the spatial data of the candidate molecule comprises determining at least the atomic species of atoms at different locations within the candidate molecule and the bonds between the atomic species and adjacent atomic species in the molecule.

4. The method of claim 3, wherein the step of determining the spatial data of the infectious agent comprises determining at least the atomic species of atoms at different locations within the infectious agent and the bonds between the atomic species and adjacent atomic species in the molecule.

5. The method of claim 4, further comprising creating a single molecular attribute numeric representation of each of a plurality of the atoms in the candidate molecule, the single molecular attribute numeric representation comprising the coordinates of the of the atom in the candidate molecule and the attributes thereof at the location.

6. The method of claim 5, further comprising creating a single molecular attribute numeric representation of each of a plurality of the atoms in the infectious agent, the single molecular attribute numeric representation comprising the coordinates of the of the atom in the infectious agent and the attributes thereof at the location.

7. The method of claim 6, further comprising combining the plurality of single molecular attribute numeric representations of the candidate module into a combining the plurality of single molecular attribute numeric representations of the candidate module into a numerical matrix representation of the candidate molecule; and
combining the plurality of single molecular attribute numeric representations of the infectious agent into a numerical matrix representation of the infectious agent.

8. The method of claim 7, further comprising:
adding a candidate molecule start token to the numerical matrix representation of the candidate molecule;
adding an infectious agent start token to the numerical matrix representation of the infectious agent.

9. The method of claim 8, further comprising combining the candidate molecule start token, the numerical matrix representation of the candidate molecule, the infectious agent start token and the numerical matrix representation of the infectious agent into a molecular spatial data matrix; and
inputting the infectious agent start token to the numerical matrix representation of the infectious agent into a transformer neural network.

10. The method of claim 9, wherein the transformer neural network comprises:
Nₓ Encoder Blocks, where x is 0 or a positive integer, and the encoder blocks are sequentially connected;
Nₓ decoder Blocks, where x is 0 or a positive integer, and the decoder blocks are sequentially connected; wherein
the output of the final encoder block is sent to each decoder block, and the output of each decoder block is sent to each decoder block between the decoder block and an output location of the sequentially connected decoder blocks.

11. The method of claim 10, wherein each encoder block comprises a multihead attention layer configured to receive multiple copies of the input to the encoder block;
a first add and normalize layer configured to receive the output of the multihead attention layer and the input to the encoder block
a first linear layer configured to receive the output of the first add and normalize layer; and
a second add and normalized layer configured to receive the output of the first add and normalize layer and the output of the first linear layer.

12. The method of claim 11, wherein each decoder block comprises:
a masked multi head attention layer configured to receive the output of the second add and normalize layer of the last of the sequentially connected encoder blocks;
a third add and normalize layer configured to receive the output of the masked attention layer and the output of the second add and normalize layer of the last of the sequentially connected encoder blocks;
a decoder multihead attention layer configured to receive the output of the second add and normalize layer of the last of the sequentially connected encoder blocks and the output of the third add and normalize layer;
a fourth add and normalize layer configured to receive the output of the decoder multihead attention layer and the output of the second add and normalize layer,
a second linear layer configured to receive the output of the fourth add and normalize layer; and
a fifth add and normalize layer configured to receive the output of the fourth add and normalize layer and the output of the second linear layer.

13. The method of claim 12, wherein the multihead attention layer comprises a plurality of scaled dot product attention layers connected in parallel; and
each scaled dot product attention layer is configured to receive at least three copies of the output of the second add and normalize layer.

14. The method of claim 10, wherein at least one encoder block comprises a multihead attention layer configured to receive multiple copies of the input to the encoder block;
a first add and normalize layer configured to receive the output of the multihead attention layer and the input to the encoder block;
a switch gate layer configured to receive the output of the first add and normalize layer, the switch gate layer comprising a router and a plurality of feed forward network experts configured to selectively receive the output of the router; and
a second add and normalized layer configured to receive output of the switch gate layer.
